(19) United States
(12) Patent Application Publication
(10) Pub. No.: US 2026/0355372 A1
(43) Pub. Date: Nov. 12, 2026

(54) REUSABLE PHARMACEUTICAL CONTAINERS AND PROCESSES OF REUSING THE SAME

(71) Applicant: CORNING INCORPORATED, CORNING, NY (US)

(72) Inventor: Robert Anthony Schaut, Horseheads, NY (US)

(21) Appl. No.: 19/134,574

(22) PCT Filed: Nov. 22, 2023
(86) PCT No.: PCT/US2023/080827

§ 371 Date: May 30, 2025

Related U.S. Application Data
(60) Provisional application No. 63/428,931, filed on Nov. 30, 2022.

(51) Int. Cl. A61L 2/04; A61L 103/20; C03C 21/00; C03C 23/00

(52) U.S. Cl. CPC A61L 2/04; C03C 21/002; C03C 23/007; C03C 23/0075; A61L 2103/20

(57) ABSTRACT

A container for reuse may include a glass, an inner surface, and an outer surface. The container may be a pharmaceutical container adapted to hold a pharmaceutical product. The container may have a retained strength of within 25% of an unused container prior to the first use. The inner surface may have a chemical durability ratio of 5 or less.

### CROSS-REFERENCE TO RELATED APPLICATIONS

This application claims the benefit of priority under 35 U.S.C. § 119 of U.S. Provisional Application Ser. No. 63/428,931 filed on Nov. 30, 2022, the content of which is relied upon and incorporated herein by reference in its entirety.

### FIELD OF THE DISCLOSURE

This disclosure relates to pharmaceutical containers, and more particularly, to reusable pharmaceutical containers and processes of reusing the same.

### BACKGROUND

Historically, glass has been used as the preferred material for packaging pharmaceuticals because of its hermeticity, optical clarity, and excellent chemical durability relative to other materials. Specifically, the glass used in pharmaceutical packaging must have adequate chemical durability so as to not affect the stability of the pharmaceutical compositions contained therein and adequate mechanical performance to prevent breakage of the packaging during processing or use. However, currently, glass pharmaceutical packaging is often used only a single time before being sent to be recycled or, more commonly, thrown away. This results in a large amount of waste being generated through the use of glass pharmaceutical packaging.

### SUMMARY

Accordingly, an ongoing need exists for glass pharmaceutical packaging that is capable of being reused and for processes for reconditioning and reusing glass pharmaceutical packaging. The present disclosure is directed to glass pharmaceutical packaging that can be reconditioned and reused and processes for reconditioning and reusing the glass pharmaceutical packaging.

According to one or more embodiments, a container for reuse may comprise a glass, an inner surface, and an outer surface. The container may be a pharmaceutical container adapted to hold a pharmaceutical product. The container may have a retained strength of within 25% of an unused container prior to the first use. The inner surface may have a chemical durability ratio of 5 or less.

According to additional embodiments, a process for reusing a used pharmaceutical container may comprise receiving a used pharmaceutical container and reconditioning the used pharmaceutical container to produce a reconditioned pharmaceutical container. Reconditioning the used pharmaceutical container may comprise washing the used pharmaceutical container with a caustic solution, washing the used pharmaceutical container with water, and depyrogenating the used pharmaceutical container to produce a reconditioned pharmaceutical container. After depyrogenating the used pharmaceutical container the level of organic and inorganic contaminants in the reconditioned pharmaceutical container may be less than USP limits.

Additional features and advantages of the embodiments described herein will be set forth in the detailed description which follows, and in part will be readily apparent to those skilled in the art from that description or recognized by practicing the embodiments described herein, including the detailed description which follows, the claims as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE DRAWINGS

FIG. 1 schematically depicts a cross section of a glass container in accordance with one or more embodiments described herein;

FIG. 2A depicts a flow chart that includes steps in a process for reusing a used pharmaceutical container, according to one or more embodiments described herein;

FIG. 2B depicts a flow chart that includes steps in a process for reusing a used pharmaceutical container, according to one or more embodiments described herein; and

FIG. 2C depicts a flow chart that includes steps in a process for reusing a used pharmaceutical container, according to one or more embodiments described herein.

### DETAILED DESCRIPTION

Specific embodiments of the present application will now be described. The disclosure may, however, be embodied in different forms and should not be construed as limited to the embodiments set forth in this disclosure. Rather, these embodiments are provided so that this disclosure will be thorough and complete, and will fully convey the scope of the subject matter to those skilled in the art.

Embodiments of the present disclosure are also directed to processes for reusing a used pharmaceutical container. The processes may include receiving a used pharmaceutical container and reconditioning the used pharmaceutical container to produce a reconditioned pharmaceutical container. Reconditioning the used pharmaceutical container may include washing the used pharmaceutical container with a caustic solution, washing the used pharmaceutical container with water, and depyrogenating the used pharmaceutical container. After depyrogenating the used pharmaceutical container to produce the reconditioned pharmaceutical container, the level of organic and inorganic contaminates in the reconditioned pharmaceutical container may be less than detectable limits.

The containers and processes for reusing containers of the present disclosure may generate less waste than the use of conventional containers and/or may reduce energy consumption and processing costs associated with melting down the glass containers. Typically, pharmaceutical containers are disposed of after only one use generating a significant amount of waste, much of which may end up in a landfill. The containers and processes of the present disclosure may allow for the reuse of pharmaceutical containers reducing the amount of waste generated from using pharmaceutical containers and accordingly reducing the amount of waste that requires disposal in a landfill.

As used herein, the term “recycle” refers to a process of melting down the glass of the pharmaceutical container and forming the molten glass into one or more glass articles.

As used herein, the term “reuse” refers to using a pharmaceutical container after the pharmaceutical container has already been used at least once before and then reconditioned without melting down the glass container and forming the molten glass into a new container.

As used herein, the term “chemical durability ratio” (CDR) refers to a measurement of the propensity for delamination of a glass container. As described in U.S. Patent Application Publication No. 2021/0080448 A1, the contents of which are incorporated herein by reference in their entirety, CDR depicts the level of heterogeneity on the internal surface of the container through the ratio of the “as-received” and “post-etch” titration values for a container.

As used herein, the term “delamination” refers to a phenomenon in which glass particles are released from the surface of the glass following a series of leaching, corrosion, and/or weathering reactions. In general, the particles are silica-rich flakes, or lamellae, of glass that originate from the interior surface of the container as a result of the leaching of modifier ions or weak network formers, such as, for example, boron, into a solution contained within the container. These flakes, or lamellae, may generally be from 1 nm to 2 microns thick with a width greater than about 50 microns. As these flakes, or lamellae, are primarily composed of silica, the flakes or lamellae, generally do not further degrade after being released from the surface of the glass.

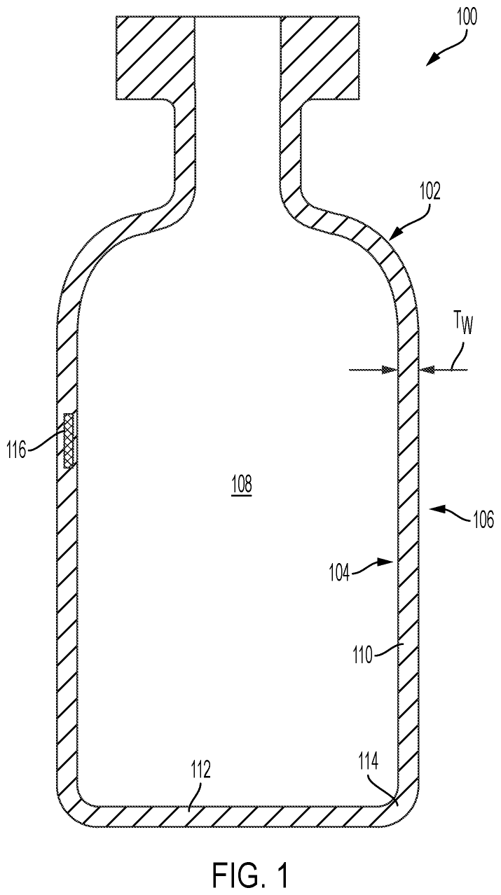

Referring now to FIG. 1, one embodiment of a container 100 for reuse is schematically depicted in cross section. The container 100 generally comprises a body 102. The body 102 extends between an inner surface 104 and an outer surface 106 and encloses an inner volume 108. In the embodiment shown in FIG. 1, the body 102 generally comprises a cylindrical wall 110 and a floor 112. The cylindrical wall 110 transitions into the floor 112 through a heel 114. The body 102 has a wall thickness Tw which extends between the inner surface 104 and the outer surface 106.

As described herein, in embodiments, the container 100 for reuse may comprise a glass, an inner surface 104, and an outer surface 106. In embodiments, the container 100 may be a pharmaceutical container. In embodiments the container 100 may be adapted to hold one or more of a pharmaceutical product, a vaccine, a biologic, a solution, or combinations of these. While the container 100 is depicted in FIG. 1 as a vial, it should be understood that the container 100 may have other form factors, including, without limitation, Vacutainers®, cartridges, syringes, bottles, flasks, phials, tubes, beakers or the like.

In embodiments, the glass may be an aluminosilicate glass composition. In embodiments, the glass may be an aluminosilicate glass composition that meets Type 1 criteria as defined in USP <660>, such as those glass compositions disclosed in U.S. Pat. No. 8,551,898 hereby incorporated by reference in its entirety, and sold by Corning® Incorporated as Valor® Glass, and those disclosed in U.S. Pat. No. 9,145,329, hereby incorporated by reference in its entirety. In embodiments, the glass may be an alkali aluminosilicate glass such as those disclosed in U.S. Pat. No. 10,640,415, entitled Lithium Containing Aluminosilicate Glasses filed Nov. 29, 2017, hereby incorporated by reference in its entirety, or those disclosed in U.S. Patent Publication No. 2020/0290920, entitled Chemically Durable Aluminosilicate Glass Compositions and Glass Articles Formed Therefrom, filed Sep. 17, 2020, hereby incorporated by reference in its entirety. In embodiments, the glass may be an aluminosilicate glass composition that has been subjected to an etching process, such as acid etching or fluoride etching, to remove deposits on the inner surface 104 of the container 100. In embodiments, the glass may be a 33 expansion borosilicate glass such as those sold by DWK Life Sciences as KIMBAL® 33 or those sold by Schott as BOROFLOAT® 33. Expansion 33 glass has a coefficient of thermal expansion of 33 and is a Type 1A glass per USP <660>. In embodiments, the glass may be a 51 expansion borosilicate glass, such as those sold by DWK Life Sciences as KIMBAL® 51 or those sold by Corning® as 51-D clear borosilicate glass tubing. Expansion 51 glass has a coefficient of thermal expansion of 51 and is a Type 1B glass per USP <660>. In embodiments, where the glass is a 33 expansion or 51 expansion borosilicate glass the outer surface 106 of the container 100 may be coated with an external coating for example suitable containers may be coated containers sold by Corning® Incorporated as Velocity®.

In embodiments, the glass may be a strengthened aluminosilicate glass. In embodiments, the strengthened aluminosilicate glass may be formed by ion-exchanging an aluminosilicate glass in a molten salt bath. The ion exchange process may be performed in an ion exchange medium under processing conditions such as, for example, those disclosed in U.S. Pat. No. 8,551,898 which is incorporated herein by reference in its entirety and those disclosed in U.S. Pat. No. 9,145,329 which is incorporated herein by reference in its entirety. However it should be understood that the ion-exchange process is not particularly limited and that other processes are contemplated herein.

Without being bound by theory it is believed that aluminosilicate glass compositions may have sufficient chemical durability, mechanical strength, and optical performance to be reused. For instance, such glass compositions may have relatively uniform surface chemistry with comparatively low heterogeneity improving the chemical durability of the container when compared to glass compositions with less uniform surface chemistry with more heterogeneity. When glass articles containing volatile species, such sodium and/or boron (e.g., glass of >0.1 mol % Na₂O and/or B₂O₃, such as >0.5 mol %, >1 mol %, >2 mol %, >4 mol %), are heated (such as during a thermo-mechanical converting process for converting glass tube into a plurality of glass containers), the sodium and/or boron may be volatilized and released from the surface of the glass. The volatized sodium and/or boron later condenses on cooler parts of the glass tube or glass container surface causing compositional heterogeneities in the glass container surface. Such compositional heterogeneities in the glass container surface can lead to reduced chemical durability and greater propensity for delamination of the glass surface.

The homogeneity of the surface concentration of the glass constituent components in the surface region of the glass is generally an indication of the propensity of the glass composition to de-laminate and shed glass particles from the inner surface 104 of the container 100. When the glass composition has a consistent surface homogeneity in the surface region (i.e., when the extrema of the surface concentration of the glass constituent components in the surface region at a discrete point A on the inner surface 104 are within +/−30% of the same constituent components in the surface region at any second discrete point B or C on the inner surface 104), the glass composition has improved resistance to delamination.

Glass containers having consistent surface homogeneity may be achieved using various techniques, including, but not limited to, acid etching at least the inner surface 104 of the body 102 of the glass container 100 or by forming the glass container from glass compositions in which the constituent components of the glass composition form species with relatively low vapor pressures (i.e., species with a low volatility) at the temperatures required to form glass tubes into the glass containers having the desired container shape. Acid etching presumes the formation of heterogeneities on the inner surface of the glass containers, and the acid etching removes the heterogeneities from the inner surface of the glass container to produce consistent surface homogeneity in the finished glass container. When the constituent components of the glass form species with relatively low vapor pressures at the reforming temperatures, the constituent components are less likely to volatilize and evaporate from the surfaces of the glass and then condense on a cooler surface of the glass. Reducing or preventing volatilization of the constituent components of the glass can enable forming a glass container with a compositionally homogenous surface over the inner surface of the glass container and through the thickness of the glass container.

In embodiments, the container 100 may be resistant to delamination following exposure to certain compositions stored in the container 100. The delamination risk of a glass container 100 may be measured using a chemical durability ratio (CDR). The method for assessing the CDR of a glass container involves (1) a hydrolytic test of the as-received surface, (2) an etching step to remove any chemical heterogeneities that may be present, and (3) a second hydrolytic test of the ‘etched’ surface. “As-received” containers are processed according to the USP <660> Surface Glass Test with one notable deviation: the filling volume is 12.5% of the brimful capacity. Due to the reduced filling volume, additional containers are needed to generate the solution volume needed for the titration. The titration volume is recorded as the “as-received” response.

Following the first USP <600> Surface Glass Test on the “as-received” containers, the “as-received” containers are then subjected to an etching process to remove the material deposited or incorporated during the converting or molding process. At least one micron (depth) of the surface is removed using a mixture of HCl/HF acids, with target concentrations of 2.3 M HF/4.6 M HCl. The containers are exposed to this solution for a minimum of 3 minutes. These conditions are sufficient for most Type 1 glass compositions, and the mass lost is measured to confirm sufficient depth of surface removal. After exposure to the target acid solution, acidic residue in the containers is removed through soaking in two room temperature water baths for 5 minutes each. Subsequently, the containers are rinsed with high purity water several times. Containers used for the “etched” response were the retained containers from the “as-received” test. After etching, a second USP <660> Surface Glass Test is conducted on the “etched” containers to measure the bulk glass response, again at the reduced 12.5% filling volume.

The “etched” containers are processed according to the USP <660> Surface Glass Test using the reduced fill volume (12.5% of the brimful capacity). The resulting titrant volume is recorded as the “etched” titrant response. A ratio of the recorded titrant volumes is calculated as follows (*at reduced volume):

$$CDR = \frac{As Received Titrant Volume}{Etched Titrant Volume} \approx \frac{< 660 > Surface Glass {Test}^{*}}{< 660 > Surface Etching {Test}^{*}}$$

The CDR value represents the risk for delamination, where containers with a uniform surface chemistry exhibit a low CDR and have the lowest risk of delamination.

As described herein, in embodiments, the container 100 may have a CDR of 5 or less. In embodiments, the inner surface 104 of the glass container 100 may have a CDR of 5 or less, such as 4 or less, 3 or less, or even 2 or less. In embodiments, the inner surface 104 may have a CDR of greater than or equal to 0.5 to less than or equal to 5. For example, the inner surface 104 may have a CDR of greater than or equal to 0.5 to less than or equal to 4.5, such as greater than or equal to 0.5 to less than or equal to 4, greater than or equal to 0.5 to less than or equal to 3.5, greater than or equal to 0.5 to less than or equal to 3, greater than or equal to 0.5 to less than or equal to 2.5, greater than or equal to 0.5 to less than or equal to 2.0, greater than or equal to 0.5 to less than or equal to 1.5, greater than or equal to 0.5 to less than or equal to 1, greater than or equal to 1 to less than or equal to 5, greater than or equal to 1 to less than or equal to 4.5, greater than or equal to 1 to less than or equal to 4, greater than or equal to 1 to less than or equal to 3.5, greater than or equal to 1 to less than or equal to 3, greater than or equal to 1 to less than or equal to 2.5, greater than or equal to 1 to less than or equal to 2, greater than or equal to 1 to less than or equal to 1.5, greater than or equal to 1.5 to less than or equal to 5, greater than or equal to 1.5 to less than or equal to 4.5, greater than or equal to 1.5 to less than or equal to 4, greater than or equal to 1.5 to less than or equal to 3.5, greater than or equal to 1.5 to less than or equal to 3, greater than or equal to 1.5 to less than or equal to 2.5, greater than or equal to 1.5 to less than or equal to 2, greater than or equal to 2 to less than or equal to 5, greater than or equal to 2 to less than or equal to 4.5, greater than or equal to 2 to less than or equal to 4, greater than or equal to 2 to less than or equal to 3.5, greater than or equal to 2 to less than or equal to 3, greater than or equal to 2 to less than or equal to 2.5, greater than or equal to 2.5 to less than or equal to 5, greater than or equal to 2.5 to less than or equal to 4.5, greater than or equal to 2.5 to less than or equal to 4, greater than or equal to 2.5 to less than or equal to 3.5, greater than or equal to 2.5 to less than or equal to 3, greater than or equal to 3 to less than or equal to 5, greater than or equal to 3 to less than or equal to 4.5, greater than or equal to 3 to less than or equal to 4, greater than or equal to 3 to less than or equal to 3.5, greater than or equal to 3.5 to less than or equal to 5, greater than or equal to 3.5 to less than or equal to 4.5, greater than or equal to 3.5 to less than or equal to 4, greater than or equal to 4 to less than or equal to 5, greater than or equal to 4 to less than or equal to 4.5, greater than or equal to 4.5 to less than or equal to 5, or any combination of these ranges. Without being bound by theory, it is believed that a CDR of greater than 5 may result in greater probability of delamination during reuse of the container 100. Containers 100 that have adequate delamination performance for single use applications may not have adequate delamination performance for reuse. It is believed that the likelihood of delamination may increase each time the container is used because each use may potentially expose the container to compositions, such as but not limited to corrosive compositions, which may increase the likelihood of delamination. It is believed that containers 100 with an inner surface 104 having a CDR of 5 or less may have adequate delamination performance even after multiple uses.

As the container 100 is used, the container 100 may lose a certain level of mechanical strength after each use. In embodiments, a used container 100 may retain a certain percentage of mechanical strength when compared to an unused container 100. In embodiments, the retained mechanical strength of the used container 100 may be within 25% of the mechanical strength of the original container prior to the first use. For example, the retained mechanical strength of the used container may be within 24% of the mechanical strength of the original container prior to the first use, such as within 22%, within 20%, within 18%, within 16%, within 14%, within 12%, within 10%, within 8%, within 6%, within 4% or even within 2% of the mechanical strength of the original container prior to the first use. In embodiments, the mechanical strength of the container 100 may be measured by quasistatic modulus of rupture testing, where the required horizontal compression, vertical compression, or internal pressure (burst pressure) required to rupture the container 100 is used to determine the mechanical strength of the container 100.

In embodiments, the container 100 may comprise a low friction coating on the outer surface 106 of the container, which may help to preserve the mechanical strength of the container 100. The outer surface coated with the low friction coating may have a coefficient of friction less than or equal to 0.7. In embodiments, the low friction coating on the outer surface 106 of the container 100 may be a low friction coating as described in U.S. Pat. No. 9,763,852, hereby incorporated by reference in its entirety. However, it should be understood that other low friction coatings are contemplated herein. Without being bound by theory it is believed that applying a low friction coating on the outer surface 106 of the container 100 may reduce the mechanical damage that occurs to the outer surface 106 of the container 100, such as abrasions, that occurs when the container 100 contacts processing equipment, handling equipment, or other containers during routine use. As mechanical damage builds up, the strength of the container 100 may decrease, which may make the container 100 less suitable for reuse. Accordingly, reducing the mechanical damage that occurs to the container 100 may improve the lifespan of the container 100 facilitating the reuse of the container 100.

As the container 100 is used, the container 100 the contents of the container 100 may be visually inspected to ensure, for instance, that delamination of the container 100 has not occurred. The container 100 may have specific optical properties to ensure that such visual inspection can occur. Optical properties may include, but are not limited to, transparency, color haze, refractive index, light scattering, or combinations of these. In embodiments the transparency throughout the visible spectrum of a used pharmaceutical container 100 may be within 10% of the transparency throughout the visible spectrum of an unused container 100. For example, the transparency throughout the visible spectrum of the used container 100 may be within 9% of the transparency throughout the visible spectrum of an unused container 100, such as within 8%, with 7%, within 6%, within 5%, within 4%, within 3%, within 2%, or even within 1% of the transparency throughout the visible spectrum of the original container prior to the first use.

In embodiments, the color haze of a used pharmaceutical container 100 may be within 10% of the color haze of an unused container 100. In embodiments, the refractive index of a used pharmaceutical container 100 may be within 10% of the refractive index of an unused container 100. In embodiments, the light scattering of a used pharmaceutical container 100 may be within 10% of the light scattering of an unused container 100. In embodiments, the container 100 may have no coloration, scattering and/or haze. In embodiments, the container 100 may have minimal scratches or other accumulated cosmetic damage which may cause inconsistencies in the appearance of the container 100.

In embodiments, the container 100 may further comprise a unique identification code. In embodiments, the unique identification code may be used to identify the manufacturing lot of the container, the prior contents of the container 100, the number of prior uses of the container 100, or combinations of these. The unique identification code may, by way of example and not limitation, be suitable to provide resolution at a level of billions of unique identifications, and may be coded into a marking by way of the shape, structure, or resonance of the marking. The marking comprising the unique identification code may be permanently affixed to or embedded in a surface of the glass. The unique identification code may directly encode part manufacturing information such as a date of manufacture, the plant of origin, and the like. The amount of information required to be encoded in any particular unique identification code will vary depending upon the requirements of the unique identification code to be employed. As but one example, the mark comprising the unique identification code may be in the form of a one-dimensional (1-D) or two-dimensional (2-D) barcode. Two-dimensional unique identification codes encoding from as few as 10 numerical digits or less to as many as 36 alphanumeric digits or more are useful for the tracking of pharmaceuticals, with unique identification codes encoding 16 alphanumeric digits being considered typical. Sixteen-digit patterns can incorporate sufficient information for most manufacturing purposes, are readily printable in machine-readable sizes within the glass. In embodiments, the unique identification code may contain information about the container, may identify the container, may redirect to a database with information about the used pharmaceutical container, or combinations of these. In embodiments, the mark comprising the unique identification code may be a quick response (QR) code. In embodiments, the mark comprising the unique identification code may only be visible when the container 100 is exposed to ultra-violet (UV) light, such as those marks disclosed in U.S. Pat. No. 10,676,240, hereby incorporated by reference in its entirety. In embodiments, the mark comprising the unique identification code may be laser etched into the glass. In some embodiments, as shown in FIG. 1, the mark comprising the unique identification code may be a laser etched marking 116 disposed between the inner surface 104 and the outer surface 106 of the container. In embodiments, the mark comprising the unique identification code may be printed onto a surface of the container 100. Suitable methods for applying the mark comprising the unique identification code through laser etching, surface printing, or both are disclosed in U.S. Pat. No. 10,676,240, incorporated by referenced in its entirety herein above. However, it should be understood that other methods of applying the mark comprising the unique identification code are contemplated herein.

In embodiments, the inner surface 104 of the container 100 may comprise a total concentration of organic and/or inorganic contaminates that is less than detectable limits. Pharmaceutical manufacturers and government agencies may require specific maximum levels of organic or inorganic contaminates in a container for use as pharmaceutical packaging. For example, the acceptable level of organic or inorganic contaminates in the container 100 may be less than detectable limits, such that no inorganic or organic contaminates can be conventionally detected within the interior volume 108 or on the inner surface 104 of the container 100. In embodiments, the container 100 may have less than 0.15 μg of any organic contaminates that can be conventionally detected within the interior volume 108 or on the inner surface 104 of the container 100. In embodiments, the container 100 may have inorganic contaminates detected within the interior volume 108 or on the inner surface 104 below the limits established in USP <232> and <233>. In embodiments, the interior surface 104 of the container 100 may have less than 10 ppm of each element detectable in a USP surface glass test extraction.

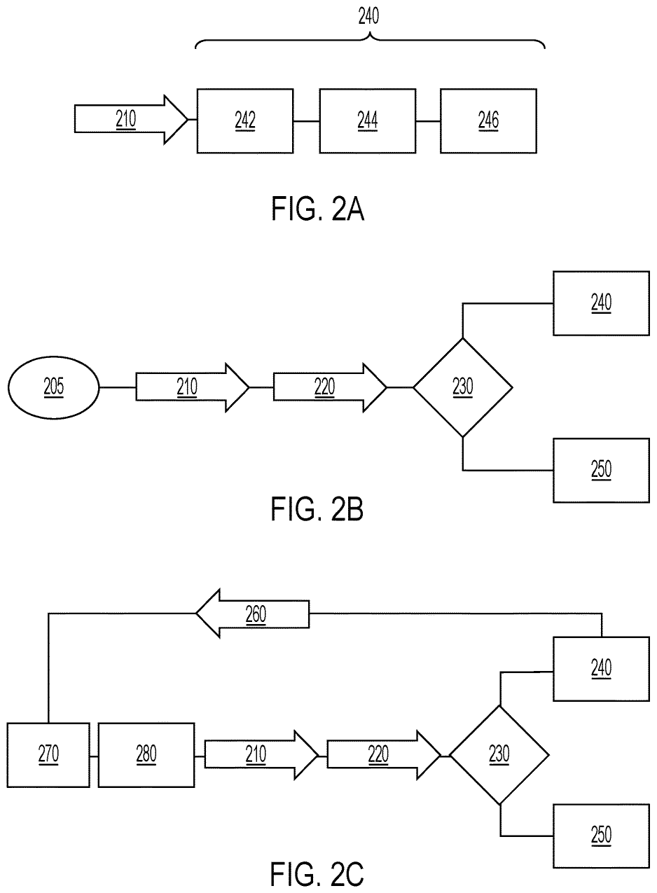

As noted above, a process for reusing a used pharmaceutical container is disclosed herein. As shown in FIG. 2A, In embodiments, the process 200 for reusing a used pharmaceutical container may comprise receiving a used pharmaceutical container (step 210) and reconditioning the used pharmaceutical container (step 240) to produce a reconditioned pharmaceutical container. In embodiments, reconditioning the used pharmaceutical container (step 240) may comprise washing the used pharmaceutical container with a caustic solution (step 242), washing the used pharmaceutical container with water (step 244), and depyrogenating the used pharmaceutical container (step 246). After depyrogenating the used pharmaceutical container (step 246) to produce the reconditioned pharmaceutical container, the level of organic and inorganic contaminants in the reconditioned pharmaceutical container may be less than detectable limits.

In embodiments, the used pharmaceutical container may be washed with a caustic solution (step 242). In embodiments, the caustic solution may have a pH of from 11 to 14, such as from 11 to 13, 11 to 12, 12 to 14, 12 to 13, 13 to 14, or any combination of these ranges. The process of claim 16, wherein the caustic solution comprises one or more metal hydroxides, metal carbonates, citrates, acetates, oxidative species, chelating species, complex-forming species, surfactants, soaps, or combinations of these. For example, the caustic solution may comprise one or more of sodium hydroxide, potassium hydroxide, calcium hydroxide, magnesium hydroxide, magnesium carbonate, calcium carbonate, sodium carbonate, potassium carbonate, sodium citrate, potassium citrate, magnesium citrate, calcium citrate, sodium acetate, potassium acetate, calcium acetate, magnesium acetate, or combinations of these. Without being bound by theory, it is believed that washing the used pharmaceutical container with a caustic solution may remove any inorganic or organic contaminates remaining on the surface of the glass from the prior use of the used pharmaceutical container.

In embodiments, washing the used pharmaceutical container with a caustic solution (step 242) may further comprise heating the caustic solution to a temperature of from 50 °C to 95 °C, such as from 50 °C to 90 °C, from 50 °C to 85 °C, from 50 °C to 80 °C, from 50 °C to 75 °C, from 50 °C to 70 °C, from 50 °C to 65 °C, from 50 °C to 60 °C, from 50 °C to 55 °C, from 55 °C to 95 °C, from 55 °C to 90 °C, from 55 °C to 85 °C, from 55 °C to 80 °C, from 55 °C to 75 °C, from 55 °C to 70 °C, from 55 °C to 65 °C, from 55 °C to 60 °C, from 60 °C to 95 °C, from 60 °C to 90 °C, from 60 °C to 85 °C, from 60 °C to 80 °C, from 60 °C to 75 °C, from 60 °C to 70 °C, from 60 °C to 65 °C, from 65° to 95 °C, from 65° to 90 °C, from 65° to 85 °C, from 65° to 80 °C, from 65° to 75 °C, from 65° to 70 °C, from 70° to 95 °C, from 70° to 90 °C, from 70° to 85 °C, from 70° to 80 °C, from 70 °C to 75 °C, from 75° to 95 °C, from 75 °C to 90 °C, from 75 °C to 85, from 75 °C to 80 °C, from 80 °C to 95 °C, from 80 °C to 90 °C, from 80 °C to 85 °C, from 85 °C to 95 °C, from 85 °C to 90 °C, from 90 °C to 95 °C, or any combination of these ranges. Without being bound by theory it is believed that caustic solutions at a temperature less than 50 °C may not as effectively remove inorganic and organic contaminates from the used pharmaceutical container as caustic solutions at temperatures greater than 50 °C.

In embodiments, reconditioning the used pharmaceutical container (step 240) may comprise washing the used pharmaceutical container with water (step 244). Washing with water in step 244 may be conducted after washing the used pharmaceutical container with the caustic solution in step 242. The water wash may remove any residual caustic solution from the surfaces of the used pharmaceutical container. In embodiments, the water for the water wash of step 244 may be at ambient temperature. In embodiments, the water for the water wash or step 244 may be heated to a temperature that is above ambient temperature, such as, for example, a temperature greater than or equal to 30 °C, greater than or equal to 40 °C, greater than or equal to 50 °C, greater than or equal to 60 °C, greater than or equal to 70 °C, greater than or equal to 80 °C, or even greater than or equal to 90 °C. The temperature of the water during the water wash step 244 may be less than 100 °C.

In embodiments, reconditioning the used pharmaceutical container (step 240) may comprise first washing the container with a caustic solution (step 242), then washing the container with water (step 244) after the caustic solution wash, and then depyrogenating the used pharmaceutical container (step 246) after the water wash. In embodiments, reconditioning the used pharmaceutical container (step 240) may further comprise exposing the container to UV light. In embodiments, reconditioning the used pharmaceutical container (step 240) may further comprise exposing the container to ozone. Without being bound by theory, it is believed that ozone exposure and UV light exposure may oxidize organic contaminates, which may beneficially assist their removal from the container.

Referring again to FIG. 2A, in embodiments, reconditioning the used pharmaceutical container (step 240) may comprise depyrogenating the used pharmaceutical container (step 246). The depyrogenating the used pharmaceutical container (step 246) may be conducted after washing the used pharmaceutical container with the caustic solution (step 242) and the water (step 244). In embodiments, depyrogenating the used pharmaceutical container may comprise heating the used pharmaceutical container to a depyrogenation temperature of from 250 °C to 400 °C, such as from 250 °C to 375 °C, from 250 °C to 350 °C, from 250 °C to 325 °C, from 250 °C to 300 °C, from 250 °C to 275 °C, from 275 °C to 400 °C, from 275 °C to 375 °C, from 275 °C to 350 °C, from 275 °C to 325 °C, from 275 °C to 300 °C, from 300 °C to 400 °C, from 300 °C to 375 °C, from 300 °C to 350 °C, from 300 °C to 325 °C, from 325 °C to 400 °C, from 325 °C to 375 °C, from 325 °C to 350 °C, from 350 °C to 400 °C, from 350 °C to 375 °C, from 375 °C to 400 °C, or any combination of these ranges.

In embodiments, depyrogenating the used pharmaceutical container (step 246) may comprise heating the used pharmaceutical container to the depyrogenation temperature and maintaining the used pharmaceutical container at the depyrogenation temperature for a time period of from about 30 seconds to about 72 hours, such as, from about 30 seconds to about 60 hours, from about 30 seconds to about 48 hours, from about 30 seconds to about 36 hours, from about 30 seconds to about 24 hours, from about 30 seconds to about 12 hours, from about 30 seconds to about 6 hours, from about 30 seconds to about 1 hour, from about 1 hour to about 72 hours, from about 1 hour to about 60 hours, from about 1 hour to about 48 hours, from about 1 hour to about 36 hours, from about 1 hour to about 24 hours, from, from about 1 hour to about 12 hours, from about 1 hour to about 6 hours, from about 6 hours to about 72 hours, from about 6 hours to about 60 hours, from about 6 hours to about 48 hours, from about 6 hours to about 36 hours, from about 6 hours to about 24 hours, from about 6 hours to about 12 hours, from about 12 hours to about 72 hours, from about 12 hours to about 60 hours, from about 12 hours to about 48 hours, from about 12 hours to about 36 hours, from about 12 hours to about 24 hours, from about 24 hours to about 72 hours, from about 24 hours to about 60 hours, from about 24 hours to about 48 hours, from about 24 hours to about 36 hours, from about 36 hours to about 72 hours, from about 36 hours to about 60 hours, from about 36 hours to about 48 hours, from about 48 hours to about 72 hours, from about 48 hours to about 60 hours, from about 60 hours to about 72 hours, or any combination of these ranges.

In embodiments, reconditioning the used pharmaceutical container 240 may comprise first washing the container with a caustic solution 242, then washing the container with water 244 after the caustic solution wash, and then depyrogenating the used pharmaceutical container 246 after the water wash.

In embodiments, the process for reusing a used pharmaceutical container may further comprise sending the reconditioned pharmaceutical container for reuse. In embodiments, the reconditioned pharmaceutical container may be sent for reuse and may be reused in the same manner as it was being used prior to reconditioning. In embodiments, the reconditioned pharmaceutical container that previously contained a particular pharmaceutical product initially may be sent for reuse with the same pharmaceutical product. In embodiments, the reconditioned pharmaceutical container may be sent for reuse and may be reused in a different manner than it was used prior to reconditioning. For instance, in embodiments, the reconditioned pharmaceutical container that was previously used to contain a first pharmaceutical product may be sent for reuse with a second pharmaceutical product that is different from the first pharmaceutical product.

Referring now to FIG. 2B, in embodiments, the process 200 for reusing the used pharmaceutical container may further comprise receiving information relating to the used pharmaceutical container (step 220) after receiving the used pharmaceutical container (step 210). The pharmaceutical container may be received (step 210) after being used (step 205). The process 200 may also further comprise determining whether to recondition and reuse the used pharmaceutical container (step 230) based on the information relating to the used pharmaceutical container. When it is determined not to recondition (step 240) and reuse the used pharmaceutical container the container may be recycled or disposed of (step 250). The information relating to the used pharmaceutical containing may include, but is not limited to, the prior contents of the used pharmaceutical container, the number of times that the used pharmaceutical container has been reused, the lot number of the used pharmaceutical container, other information on the used pharmaceutical container, or combinations thereof.

In embodiments, the information relating to the used pharmaceutical container may include the prior contents of the used pharmaceutical container, and the decision whether to recondition and reuse the used pharmaceutical container may be based wholly or in part on the prior contents of the used pharmaceutical container. In embodiments, the prior contents of the used pharmaceutical container may prevent the container from being reused. For instance, containers used for human blood and blood components or containers used for radioactive materials may not be allowed to be reused by government regulation and must instead be recycled or disposed of. In other embodiments, the prior contents of the used pharmaceutical container may allow the reuse of the used pharmaceutical container and the used pharmaceutical container may be reconditioned.

In embodiments, the used pharmaceutical container may comprise the unique identification code. In these embodiments, the process for reusing the used pharmaceutical container may further comprise reading the unique identification code of the used pharmaceutical container; obtaining a total number of reuse cycles, an identification of prior contents for the used pharmaceutical container, or both from a used container database using the unique identification code; and determining whether to reuse the pharmaceutical container based on the total number of reuse cycles of the used pharmaceutical container, the identification of the prior contents of the used pharmaceutical container or both. The term “used container database” refers to any database or collection of information linking the unique identification code of the used pharmaceutical container with information about the used pharmaceutical container, such as but not limited to prior contents, number of reuse cycles, lot number, or other information. In embodiments, when the total number of reuse cycles and the identification of the prior contents both indicate that reuse of the used pharmaceutical container is permissible, the process may include reconditioning the used pharmaceutical container to produce a reconditioned pharmaceutical container. In embodiments, when either the total number of reuse cycles or the identification of the prior contents indicates that the reuse of the used pharmaceutical container is not permissible, the process may include recycling or disposing of the used pharmaceutical container.

In embodiments, determining whether to reuse the used pharmaceutical container comprises comparing the total number of reuse cycles against a threshold number of reuse cycle, and when the total number of reuse cycles is greater than the threshold number not permitting the reuse of the pharmaceutical container. As used herein, the term “reuse cycle” refers to using the pharmaceutical container, reconditioning the pharmaceutical container, and sending the pharmaceutical container for use again. In embodiments, the threshold number may be less than or equal to 200 reuse cycles, such as, less than or equal to 175 reuse cycles, less than or equal to 150 reuse cycles, less than or equal to 125 reuse cycles, less than or equal to 100 reuse cycles, less than or equal to 75 reuse cycles, less than or equal to 50 reuse cycles, or even less than or equal to 25 reuse cycles. In embodiments, the threshold number may be greater than 200 reuse cycles. In embodiments, the threshold number may be greater than or equal to 1.

Without being bound by theory it is believed that the pharmaceutical container may include small amounts of physical and/or chemical damage during normal use of the pharmaceutical container during each reuse cycle. When reusing the pharmaceutical container, these small amounts of physical and/or chemical damage may build up over time, which may cause the container to no longer be suitable for reuse. Tracking the number of reuse cycles may allow used pharmaceutical containers to be removed from service at an appropriate time to reduce the risk of failure of the used pharmaceutical containers. It is further believed that a physical inspection of a pharmaceutical container may not, on its own, correctly identify a need to remove the container from service. Accordingly tracking the total number of reuse cycles may allow for a more accurate determination of whether a used pharmaceutical container should be reconditioned for reuse or sent for recycling or disposal.

In embodiments, determining whether to reuse the used pharmaceutical container may comprise comparing the identification of the prior contents against a list of materials for which the pharmaceutical containers cannot be reused. In embodiments, the list of materials for which pharmaceutical containers cannot be reused comprises materials with radioactive components, materials derived from human blood products, cytotoxic compounds, other incompatible compounds, or combinations thereof. It is contemplated that the list of materials for which pharmaceutical containers cannot be reused may change with regulatory changes or with changing scientific knowledge as specific compounds may be added or removed from the list.

In embodiments, determining whether to reuse the used pharmaceutical container further comprises inspecting the used pharmaceutical container to determine whether specific optical properties have been maintained, and when those optical properties have not been maintained disposing of or recycling the used pharmaceutical container. During the container's use as a pharmaceutical container the contents of the container may be visually inspected to ensure, for example, that delamination of the container has not occurred. The container may have specific optical properties to ensure that such visual inspection can occur. Optical properties may include, but are not limited to, transparency, haze, coloration, light scattering, refractive index, and combinations of these. In embodiments, the optical property of the used pharmaceutical container may be within 10% of the optical property of an unused container, where the optical property can be any one or a combination of transparency, haze, color, light scattering, or refractive index. In embodiments the transparency throughout the visible spectrum of a used pharmaceutical container may be within 10% of the transparency throughout the visible spectrum of an unused container.

In embodiments, determining whether to reuse the used pharmaceutical container further comprises checking the used pharmaceutical container against a used pharmaceutical container database to determine if the used pharmaceutical container is part of a manufacturing lot flagged for removal from service, and when the used pharmaceutical container is part of a manufacturing lot flagged for removal from service recycling or disposing of the used pharmaceutical container. As described herein, the unique identification code may in some embodiments contain information regarding the manufacturing of the pharmaceutical container such as a date of manufacture, the plant of origin, and the like. This may allow targeted container lots to be removed from service. If the used pharmaceutical container's manufacturing information was not able to be determined for each container flagging a manufacturing lot for removal may require containers not part of that manufacturing lot to be removed from service to ensure all containers flagged for removal were removed potentially increasing the waste generated from flagging the manufacturing lot.

Now referring to FIG. 2C, the steps in the life cycle of a pharmaceutical container are graphically depicted. Starting with step 270 the container may be filled with pharmaceutical product, a vaccine, a biologic, a foodstuff, or a solution. The container may then be used as pharmaceutical container (step 280). The used pharmaceutical container is then received in step 210 and information about the container is received in step 220. Using the received information, in step 230 it is determined whether to recondition the container (step 240) for reuse or to dispose of or recycle the container (step 250). A container that is reconditioned (step 240) is then sent for reuse in step 260. A container may go through as many as 20 cycles starting from the initial use, before it is disposed of or recycled (step 250)>.

It will be apparent to those skilled in the art that various modifications and variations can be made to the embodiments described herein without departing from the spirit and scope of the claimed subject matter. Thus it is intended that the specification cover the modifications and variations of the various embodiments described herein provided such modification and variations come within the scope of the appended claims and their equivalents.

1. A container for reuse, the container comprising a glass, an inner surface, and an outer surface, wherein the container is a pharmaceutical container adapted to hold a pharmaceutical product, and wherein:
the container has a retained strength of within 25% of a strength of an unused container prior to the first use; and
the inner surface has a chemical durability ratio (CDR) of 5 or less.

2. The container of claim 1, wherein the inner surface has a CDR of 4 or less.

3. The container of claim 1, wherein the inner surface has a CDR of greater than or equal to 0.5 to less than or equal to 5.

4. The container of claim 1, wherein the outer surface has a retained strength of within 10% of an unused container prior to the first use.

5. The container of claim 1, further comprising a unique identification code.

6. The container of claim 5, wherein the unique identification code is a quick response (QR) code.

7. The container of claim 5, wherein the unique identification code is only visible when the container is exposed to ultra-violet (UV) light.

8. The container of claim 5, wherein the unique identification code is laser etched into the glass.

9. The container of claim 5, wherein the unique identification code is printed onto a surface of the container.

10. The container of claim 1, wherein the inner surface of the container comprises a total concentration of organic and inorganic contaminates that is less than USP limits.

11. The container of claim 1, wherein the glass is an aluminosilicate glass.

12. The container of claim 11, wherein the glass is a strengthened aluminosilicate glass.

13. The container of claim 12, wherein the strengthened aluminosilicate glass is formed by ion-exchanging an aluminosilicate glass in a molten salt bath.

14. The container of claim 1, wherein the container comprises a low friction coating on the outer surface of the container.

15. The container of claim 12, wherein the pharmaceutical product comprises a medicament, a vaccine, a biologic, a solution, or combinations thereof.

16. A process for reusing a used pharmaceutical container, the process comprising:
receiving a used pharmaceutical container; and
reconditioning the used pharmaceutical container to produce a reconditioned pharmaceutical container, wherein reconditioning the used pharmaceutical container comprises:
washing the used pharmaceutical container with a caustic solution;
washing the used pharmaceutical container with water; and
depyrogenating the used pharmaceutical container to produce a reconditioned pharmaceutical container, wherein after depyrogenting the used pharmaceutical container the level of organic and inorganic contaminants in the reconditioned pharmaceutical container is less than USP limits.

17. The process of claim 16, further comprising sending the reconditioned pharmaceutical container for reuse.

18. The process of claim 16, wherein the caustic solution has a pH of from 11 to 14.

19. The process of claim 16, wherein the washing the used pharmaceutical container with a caustic solution further comprises heating the caustic solution to a temperature of from 50 °C to 95 °C.

20. The process of claim 16, wherein the caustic solution comprises one or more metal hydroxides, metal carbonates, citrates, acetates, oxidative species, chelating species, complex-forming species, surfactants, soaps, or combinations of these.

21. The process of claim 16, wherein the depyrogenating the used pharmaceutical container comprises heating the used pharmaceutical container to a temperature of from 250 °C to 400 °C for a time period from about 30 seconds to about 72 hours.

22. The process of claim 16, further comprising:
receiving information relating to prior contents of the used pharmaceutical container; and
determining whether to recondition and reuse the used pharmaceutical container based on the information relating to the prior contents of the used pharmaceutical container.

23. The process of claim 16, wherein the used pharmaceutical container comprises a unique identification code, and the process further comprises:
reading the unique identification code of the used pharmaceutical container;
obtaining a total number of re-use cycles, an identification of prior contents for the used pharmaceutical container, or both from a used container database; and
determining whether to re-use the pharmaceutical container based on the total number of re-use cycles of the used pharmaceutical container, the identification of the prior contents of the used pharmaceutical container, or both.

24. The process of claim 23, comprising obtaining the total number of re-use cycles and the identification of the prior contents of the used pharmaceutical container from the used container database, wherein:
when the total number of re-use cycles and the identification of the prior contents permits re-use of the pharmaceutical container, reconditioning the used pharmaceutical container to produce a reconditioned pharmaceutical container; and
when either the total number of re-use cycles or the identification of the prior contents does not permit re-use of the pharmaceutical container, recycling or disposing of the used pharmaceutical container.

25. The process of claim 23, wherein determining whether to re-use the used pharmaceutical container comprises comparing the total number of re-use cycles against a threshold number of re-use cycles, and when the total number of re-use cycles is greater than the threshold number not permitting the re-use of the pharmaceutical container.

26. The process of claim 25, wherein the threshold number of re-use cycles is less than or equal to 20.

27. The process of claim 23, wherein determining whether to re-use the used pharmaceutical container comprises comparing the identification of the prior contents against a list of materials for which the used pharmaceutical containers cannot be re-used.

28. The process of claim 27, wherein the list of materials for which the used pharmaceutical containers cannot be re-used comprises materials with radioactive components, materials derived from human blood products, cytotoxic compounds, or combinations thereof.

29. The process of claim 23, wherein determining whether to re-use the used pharmaceutical container further comprises inspecting the used pharmaceutical container to determine whether the transparency throughout the visible spectrum of the used pharmaceutical container is within 10% of the transparency throughout the visible spectrum of an unused pharmaceutical container prior to a first use and whether the used pharmaceutical container has any disqualifying cosmetic defects; and
when the transparency throughout the visible spectrum of the used pharmaceutical container is not within 10% of the transparency throughout the visible spectrum of an unused pharmaceutical container or the used pharmaceutical container exhibits one or more disqualifying cosmetic defects, disposing of or recycling the used pharmaceutical container.

30. The process of claim 23, wherein determining whether to re-use the used pharmaceutical container further comprises:
checking the used pharmaceutical container against a used pharmaceutical container database to determine if the used pharmaceutical container is part of a manufacturing lot flagged for removal from service; and
when the pharmaceutical container is part of a manufacturing lot flagged for removal from service recycling or disposing of the used pharmaceutical container.
